(19) United States
(12) Patent Application Publication
(10) Pub. No.: US 2007/0060599 A1
(43) Pub. Date: Mar. 15, 2007

(54) Crystalline forms of [1S-(1alpha, 3alpha, 4beta)]-2-amino-1,9-dihydro-9-[4-hydroxy-3-(hydroxymethyl)-2-methylenecyclopentyl]-6H-purin-6-one

(76) Inventors: John D. DiMarco, East Brunswick, NJ (US); Jack Z. Gougoutas, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/222,597

(22) Filed: Sep. 9, 2005

(51) Int. Cl. A61K 31/522; C07D 473/04

(52) U.S. Cl. 514/263.37; 544/276

(57) ABSTRACT

The present invention relates to crystalline forms containing [1S-(1α,3α,4β)]-2-amino-1,9-dihydro-9-[4-hydroxy-3-(hydroxymethyl)-2-methylenecyclopentyl]-6H-purin-6-one represented by formula I; 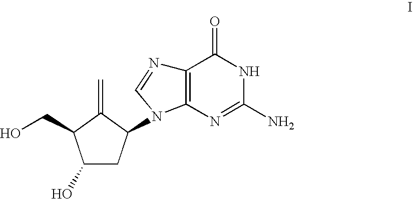 processes for the production thereof; pharmaceutical compositions thereof; methods for preparing the pharmaceutical composition; and the use of these crystalline forms in the treatment of hepatitis B viral infections.

### FIELD OF THE INVENTION

The present invention relates to crystalline forms of [1S-(1α,3α,4β)]-2-amino-1,9-dihydro-9-[4-hydroxy-3-(hydroxymethyl)-2-methylenecyclopentyl]-6H-purin-6-one; processes for the production thereof; pharmaceutical compositions thereof; methods for preparing the pharmaceutical composition; and methods for treating hepatitis B virus infection and/or co-infections.

### BACKGROUND OF THE INVENTION

Entecavir, [1S-(1α,3α,4β)]-2-amino-1,9-dihydro-9-[4-hydroxy-3-(hydroxymethyl)-2-methylenecyclopentyl]-6H-purin-6-one monohydrate, is currently being used as a drug for treating hepatitis B viral infections.

Entecavir and its use as an antiviral agent are described by Zahler et al. in U.S. Pat. No. 5,206,244, by Colonno et al. in U.S. Pat. No. 6,627,224, and by Desai et al. in US20030190334. Improved processes of preparing entecavir are described by Bisacchi et al., in WO 98/09964; by Pendri et al., in WO2004/052310 and US20040192912; and by Zhou et al. in commonly assigned and co-pending U.S. patent application Ser. No. 11/143,268, filed on Jun. 2, 2005. The disclosure of each of the foregoing patents or patent applications is herein incorporated by reference in its entirety.

The discussion of the background to the invention herein is included to explain the context of the invention. This is not to be taken as an admission that any of the material referred to was prior art as at the priority date of any of the claims.

### BRIEF DESCRIPTION OF THE DRAWINGS

FIG. 1 is a powder x-ray diffraction pattern (simulated and observed) for Form N-2 of the [1S-(1α,3α,4β)]-2-amino-1,9-dihydro-9-[4-hydroxy-3-(hydroxymethyl)-2-methylenecyclopentyl]-6H-purin-6-one.

FIG. 2 is a DSC thermogram for Form N-2 of the [1S-(1α,3α,4β)]-2-amino-1,9-dihydro-9-[4-hydroxy-3-(hydroxymethyl)-2-methylenecyclopentyl]-6H-purin-6-one.

FIG. 3 is a TGA curve for Form N-2 of the [1S-(1α,3α,4β)]-2-amino-1,9-dihydro-9-[4-hydroxy-3-(hydroxymethyl)-2-methylenecyclopentyl]-6H-purin-6-one.

FIG. 4 is a powder x-ray diffraction pattern for Form IP.3-4 of the [1S-(1α,3α,4β)]-2-amino-1,9-dihydro-9-[4-hydroxy-3-(hydroxymethyl)-2-methylenecyclopentyl]-6H-purin-6-one.

### SUMMARY OF THE INVENTION

In accordance with the present invention, there is provided the [1S-(1α,3α,4β)]-2-amino-1,9-dihydro-9-[4-hydroxy-3-(hydroxymethyl)-2-methylenecyclopentyl]-6H-purin-6-one represented by the compound of formula I in crystalline form:

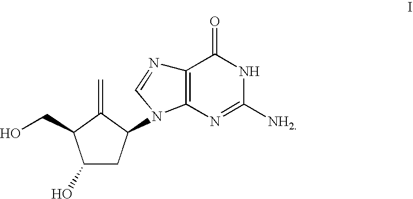

The present invention is further directed to crystalline form containing the compound of formula I, designated as Form N-2 and Form IP.3-4, as well as mixtures thereof. The present invention further pertains to processes for the production of the polymorphs; pharmaceutical compositions thereof; methods for preparing the pharmaceutical composition; and the use of these crystalline forms in the treatment of hepatitis B viral infections.

### DETAILED DESCRIPTION OF THE INVENTION

The present invention provides a crystalline form of [1S-(1α,3α,4β)]-2-amino-1,9-dihydro-9-[4-hydroxy-3-(hydroxymethyl)-2-methylenecyclopentyl]-6H-purin-6-one, depicted herein below as the compound of formula I. The invention also provides a crystalline form of [1S-(1α,3α,4β)]-2-amino-1,9-dihydro-9-[4-hydroxy-3-(hydroxymethyl)-2-methylenecyclopentyl]-6H-purin-6-one which is substantially pure, i.e., its purity greater than about 90%.

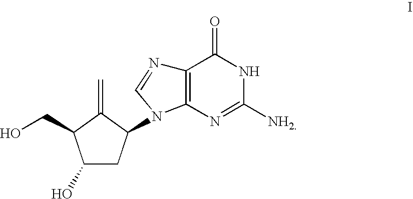

The crystalline forms of the instant invention can be characterized using Single Crystal Data, Powder X-Ray Diffraction (PXRD), Differential Scanning Calorimetry (DSC), and Thermogravimetric Analysis (TGA). It is to be understood that numerical values described and claimed herein are approximate. Variation within the values may be attributed to equipment calibration, equipment errors, purity of the materials, crystals size, and sample size, among other factors. In addition, variation may be possible while still obtaining the same result. For example, X-ray diffraction values are generally accurate to within ±0.2 degrees and intensities (including relative intensities) in an X-ray diffraction pattern may fluctuate depending upon measurement conditions employed. Similarly, DSC results are typically accurate to within about 2° C. Consequently, it is to be understood that the crystalline forms of the instant invention are not limited to the crystalline forms that provide characterization patterns (i.e., one or more of the PXRD, DSC, and TGA) completely identical to the characterization patterns depicted in the accompanying Figures disclosed herein. Any crystalline forms that provide characterization patterns substantially the same as those described in the accompanying Figures fall within the scope of the present invention. The ability to ascertain substantially the same characterization patterns is within the purview of one of ordinary skill in the art.

In one aspect of the invention, there is provided a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form N-2, which exhibits unit cell parameters approximately equal to the following at about 22° C.:

| Cell dimensions | a = 7.524(1) Å, |
|---|---|
| | b = 7.524(1) Å, |
| | c = 43.970(5) Å, |
| | Volume = 2489(1) A³, |
| Space group | P43212, |
| Molecules/unit cell | 8, |
| Density (calculated) (g/cm³) | 1.480. |

Table 1 further illustrates the unit cell data for Form N-2 crystals and Table 2 shows the fractional atomic coordinates for the asymmetric unit of Form N-2 crystals at about 22° C.

TABLE 1

| Form | T | a (Å) | b (Å) | c (Å) | α° | β° | γ° | Z′ | SG | Vₘ | R | D_{calc} |
|---|---|---|---|---|---|---|---|---|---|---|---|---|
| N-2 | 22 | 7.524 (1) | 7.524 (1) | 43.970 (5) | 90 | 90 | 90 | 1 | P4₃2₁2 | 311 | .04 | 1.480 |

T = temp (° C.) for the crystallographic data.
Z′ = number of drug molecules per asymmetric unit
Vₘ = V (unit cell)/(Z drug molecules per cell)
R = residual index (I > 3sigma (I))
D_{calc} = density of crystal calculated
SG = space group

TABLE 2

| Atom | X | Y | Z |
|---|---|---|---|
| O1 | 0.667054 | 0.068305 | −0.106759 |
| O2 | 0.571618 | 0.116352 | 0.099202 |
| N3 | 0.662231 | 0.651071 | −0.077612 |
| N4 | 0.700851 | 0.193505 | −0.004899 |
| N5 | 0.671599 | 0.354600 | −0.090095 |
| N6 | 0.689785 | 0.448266 | −0.038502 |
| N7 | 0.703595 | −0.030067 | −0.038659 |
| O8 | 0.966911 | 0.087121 | 0.119548 |
| C9 | 0.696989 | 0.132596 | −0.053543 |
| C10 | 0.676487 | 0.483243 | −0.067785 |
| C11 | 0.679224 | 0.171396 | −0.084865 |
| C12 | 0.703921 | 0.285220 | 0.024643 |
| C13 | 0.696218 | 0.271804 | −0.033061 |
| C14 | 0.706100 | 0.014087 | −0.009683 |
| C15 | 0.567168 | 0.21124 | 0.047311 |
| C16 | 0.882274 | 0.264152 | 0.040554 |
| C17 | 0.675486 | 0.119954 | 0.072150 |
| C18 | 0.848985 | 0.225864 | 0.073877 |
| C19 | 1.004899 | 0.131649 | 0.088687 |
| C20 | 1.035968 | 0.268005 | 0.026482 |
| H21 | 0.7132 | −0.0821 | 0.0086 |
| H22 | 0.6630 | 0.3981 | −0.1134 |
| H23 | 0.6772 | 0.4237 | 0.0210 |
| H24 | 0.4897 | 0.3183 | 0.0568 |
| H25 | 0.4805 | 0.1175 | 0.0362 |
| H26 | 0.7059 | −0.0144 | 0.0651 |
| H27 | 0.8313 | 0.3467 | 0.0868 |
| H28 | 1.1205 | 0.2170 | 0.0879 |
| H29 | 1.0334 | 0.0102 | 0.0763 |
| H30 | 1.1587 | 0.2518 | 0.0396 |
| H31 | 1.0436 | 0.2844 | 0.0023 |
| H32 | 0.9959 | 0.1920 | 0.1330 |
| H33 | 0.6151 | 0.0163 | 0.1127 |
| H34 | 0.6810 | 0.7522 | −0.0637 |
| H35 | 0.6712 | 0.6813 | −0.0991 |

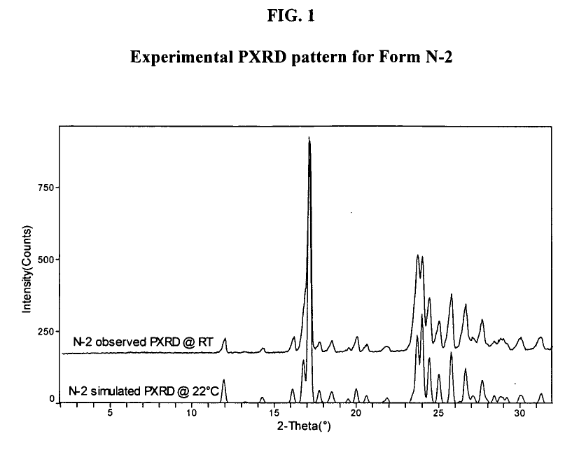

In another aspect of the invention, there is provided a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form N-2, which exhibits an PXRD pattern substantially the same as that depicted in FIG. 1, comprising characteristic diffraction peak positions (degrees 2θ) @ RT, based on a high quality pattern collected with a diffractometer (CuKα) with a spinning capillary with 2θ calibrated with a NIST other suitable standard: 11.9±0.1, 16.1±0.1, 16.8±0.1, 17.2±0.1, 20.0±0.1, 23.7±0.1, 24.0±0.1, 24.4±0.1, and 25.0±0.1.

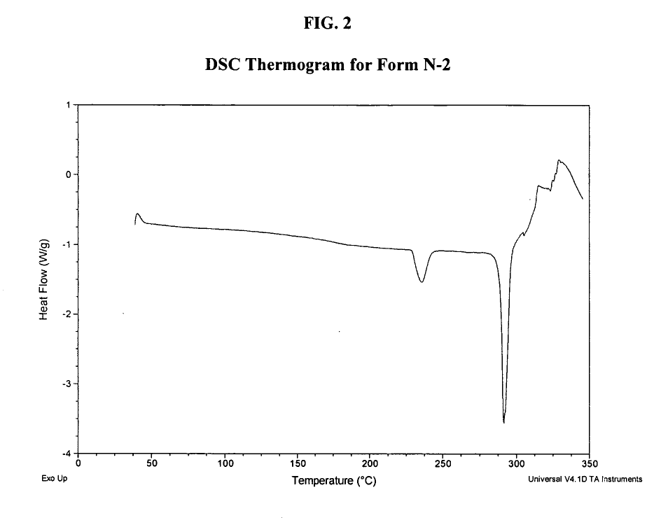

In yet another aspect, the invention provides a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form N-2, which exhibits a differential scanning calorimetry (DSC) thermogram having an endotherm typically in the range 228-245° C. The invention also provides a Form N-2 crystal that exhibits a DSC thermogram substantially the same as shown in FIG. 2.

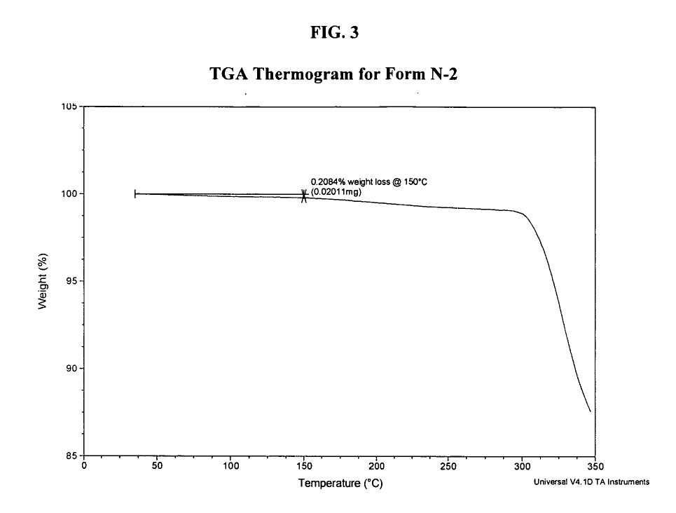

In yet another aspect, the invention provides a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form N-2, which exhibits a thermogravimetric analysis (TGA) curve having negligible weight loss up to about 150° C., in accordance to a neat form. The invention also provides a Form N-2 crystal that exhibits a TGA curve substantially the same as shown in FIG. 3.

In a further aspect, the invention provides a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form IP.3-4, which is a monoclinic solvate that contains three molecules of [1S-(1α,3α,4β)]-2-amino-1,9-dihydro-9-[4-hydroxy-3-(hydroxymethyl)-2-methylene cyclopentyl]-6H-purin-6-one, and one molecule of isopropanol per asymmetric unit. The Form IP.3-4 exhibits unit cell parameters approximately equal to the following at about −40° C.:

| Cell dimensions | a = 12.612(2) Å, |
|---|---|
| | b = 11.199(2) Å, |
| | c = 16.070(2) Å, |
| | β = 106.72(1) °, |
| | Volume = 2173.8(1) A³, |
| Space group | P21, |
| Molecules of Compound I/unit cell | 6, |
| Molecules of Isopropanol/unit cell | 2, |
| Density (calculated) (g/cm³) | 1.363. |

Table 3 further illustrates the unit cell data for Form IP.3-4 crystals and Table 4 shows the fractional atomic coordinates for the asymmetric unit of Form IP.3-4 crystals at about −40° C.

TABLE 3

| Form | T | a (Å) | b (Å) | c (Å) | α° | β° | γ° | Z′ | SG | Vₘ | R | D_{calc} |
|---|---|---|---|---|---|---|---|---|---|---|---|---|
| IPA.3-4 | −40 | 12.612 (2) | 11.199 (2) | 16.070 (2) | 90 | 106.72 (1) | 90 | 3 | P2₁ | 362 | .07 | 1.363 |

T = temp (° C.) for the crystallographic data.
Z′ = number of drug molecules per asymmetric unit
Vₘ = V (unit cell)/(Z drug molecules per cell)
R = residual index (I > 3sigma (I))
D_{calc} = density of crystal calculated
SG = space group

TABLE 4

| Atom | X | Y | Z |
|---|---|---|---|
| O1 | 0.3455 | 0.6778 | 0.3033 |
| C2 | 0.0971 | 0.8297 | 0.2553 |
| C3 | 0.0945 | 0.4982 | 0.2657 |
| O4 | 0.4077 | −0.0863 | 0.2953 |
| C5 | 0.0098 | 0.3749 | 0.4934 |
| C6 | 0.4434 | −0.0558 | 0.3774 |
| C7 | 0.1602 | 0.1368 | 0.4350 |
| C8 | 0.6360 | 0.4386 | 0.0954 |
| C9 | −0.0250 | 0.6269 | 0.3292 |
| O10 | 0.1749 | 0.9083 | 0.2603 |
| N11 | 0.0629 | 0.4835 | 0.5175 |
| C12 | 0.1652 | 0.5098 | 0.5155 |
| O13 | 0.5976 | 0.2041 | 0.5038 |
| C14 | 0.5219 | 0.1093 | 0.4895 |
| C15 | 0.3650 | 0.1595 | 0.3674 |
| C16 | 0.2919 | 0.1657 | 0.2889 |
| C17 | 0.3520 | 0.2177 | 0.4488 |
| C18 | 0.4231 | 0.1415 | 0.5217 |
| C19 | 0.4744 | 0.0886 | 0.3920 |
| O20 | 0.7306 | 0.4833 | 0.3779 |
| C21 | 0.6760 | 0.3810 | 0.3297 |
| N22 | 0.2369 | 0.2279 | 0.4498 |
| N23 | 0.0662 | 0.1708 | 0.4479 |
| C24 | 0.5539 | 0.4096 | 0.2945 |
| N25 | 0.2026 | 0.6241 | 0.5374 |
| O26 | −0.0880 | 0.3590 | 0.4930 |
| C27 | 0.0828 | 0.2888 | 0.4728 |
| C28 | 0.1882 | 0.3246 | 0.4742 |
| N29 | 0.2360 | 0.4344 | 0.4939 |
| N30 | 0.0234 | 0.4844 | 0.1758 |
| C31 | −0.0158 | 0.3780 | 0.1328 |
| N32 | −0.0874 | 0.5337 | 0.0450 |
| N33 | 0.0105 | 0.2672 | 0.1660 |
| N34 | −0.0242 | 0.0669 | 0.1321 |
| C35 | −0.0412 | 0.1866 | 0.1100 |
| C36 | −0.0828 | 0.4106 | 0.0543 |
| N37 | −0.1145 | 0.2098 | 0.0303 |
| C38 | −0.0237 | 0.5739 | 0.1182 |
| C39 | −0.1404 | 0.3214 | −0.0044 |
| O40 | −0.2074 | 0.3355 | −0.0767 |
| C41 | 0.2157 | 0.5132 | 0.2724 |
| C42 | 0.2302 | 0.6484 | 0.2627 |
| C43 | 0.0625 | 0.6128 | 0.3034 |
| C44 | 0.1535 | 0.7064 | 0.3102 |
| C45 | 0.3628 | 0.5669 | 0.1166 |
| N46 | 0.2676 | 0.5336 | 0.0629 |
| C47 | 0.3741 | 0.3720 | 0.1133 |
| C48 | 0.2733 | 0.4099 | 0.0630 |
| N49 | 0.4105 | 0.2550 | 0.1268 |
| N50 | 0.4314 | 0.4720 | 0.1485 |
| N51 | 0.2292 | 0.2100 | 0.0390 |
| C52 | 0.3323 | 0.1771 | 0.0889 |
| N53 | 0.3537 | 0.0602 | 0.0972 |
| C54 | 0.1929 | 0.3280 | 0.0189 |
| O55 | 0.1007 | 0.3445 | −0.0324 |
| C56 | 0.5430 | 0.4787 | 0.2122 |
| C57 | 0.6312 | 0.4272 | 0.1758 |
| C58 | 0.7152 | 0.3599 | 0.2498 |
| O59 | 0.6204 | 0.1671 | 0.2213 |
| C60 | 0.7228 | 0.2268 | 0.2279 |
| O61 | 0.5656 | −0.0619 | 0.2058 |
| C62 | 0.6336 | −0.1557 | 0.1981 |
| C63 | 0.6242 | −0.1943 | 0.1186 |
| C64 | 0.7391 | −0.1449 | 0.2523 |
| H65 | −0.0680 | 0.0006 | 0.0856 |
| H66 | 0.0305 | 0.0403 | 0.1933 |
| H67 | −0.0090 | 0.6699 | 0.1329 |
| H68 | −0.1535 | 0.1344 | −0.0095 |
| H69 | 0.0835 | 0.4191 | 0.3018 |
| H70 | 0.2681 | 0.4818 | 0.3337 |
| H71 | 0.2368 | 0.4656 | 0.2200 |
| H72 | 0.2058 | 0.6741 | 0.1948 |
| H73 | 0.0355 | 0.8648 | 0.2858 |
| H74 | 0.0578 | 0.8083 | 0.1898 |
| H75 | 0.5620 | 0.0314 | 0.5230 |
| H76 | 0.3073 | 0.1211 | 0.2340 |
| H77 | 0.2168 | 0.2190 | 0.2789 |
| H78 | 0.5367 | 0.1128 | 0.3586 |
| H79 | 0.3819 | −0.0763 | 0.4091 |
| H80 | 0.5185 | −0.1070 | 0.4075 |
| H81 | 0.3835 | 0.3090 | 0.4537 |
| H82 | 0.6864 | 0.3045 | 0.3718 |
| H83 | 0.7458 | 0.2209 | 0.1672 |
| H84 | 0.7906 | 0.1862 | 0.2789 |
| H85 | 0.7984 | 0.3976 | 0.2606 |
| H86 | 0.5056 | 0.3281 | 0.2798 |
| H87 | 0.5262 | 0.4609 | 0.3414 |
| H88 | 0.5601 | 0.5739 | 0.2305 |
| H89 | 0.3786 | 0.0616 | 0.5307 |
| H90 | 0.4484 | 0.1900 | 0.5823 |
| H91 | 0.1763 | 0.0473 | 0.4139 |
| H92 | −0.0431 | 0.7111 | 0.3567 |
| H93 | −0.0827 | 0.5525 | 0.3269 |
| H94 | 0.2024 | 0.7306 | 0.3758 |
| H95 | 0.3856 | 0.6614 | 0.1340 |
| H96 | 0.5525 | 0.2193 | 0.1812 |
| H97 | 0.8047 | 0.4538 | 0.4273 |
| H98 | 0.1574 | 0.6889 | 0.5258 |
| H99 | 0.2646 | 0.6471 | 0.5196 |
| H100 | 0.7024 | 0.3988 | 0.0744 |
| H101 | 0.5719 | 0.4887 | 0.0472 |
| H102 | 0.6094 | 0.0209 | 0.2037 |
| H103 | 0.4743 | −0.0771 | 0.2648 |
| H104 | 0.6329 | 0.2162 | 0.5740 |
| H105 | 0.2017 | 0.9020 | 0.2027 |
| H106 | 0.3576 | 0.7725 | 0.2962 |
| H107 | 0.0173 | 0.5526 | 0.5406 |
| H108 | 0.2893 | −0.0043 | 0.0665 |
| H109 | 0.4339 | 0.0279 | 0.1343 |
| H110 | 0.1709 | 0.1388 | 0.0114 |
| H113 | 0.6813 | −0.2671 | 0.1215 |
| H112 | 0.5406 | −0.2250 | 0.0891 |
| H114 | 0.6430 | −0.1225 | 0.0803 |
| H115 | 0.7880 | −0.2203 | 0.2434 |
| H117 | 0.7758 | −0.0634 | 0.2375 |
| H116 | 0.7365 | −0.1423 | 0.3189 |
| H111 | 0.6158 | −0.2298 | 0.2349 |

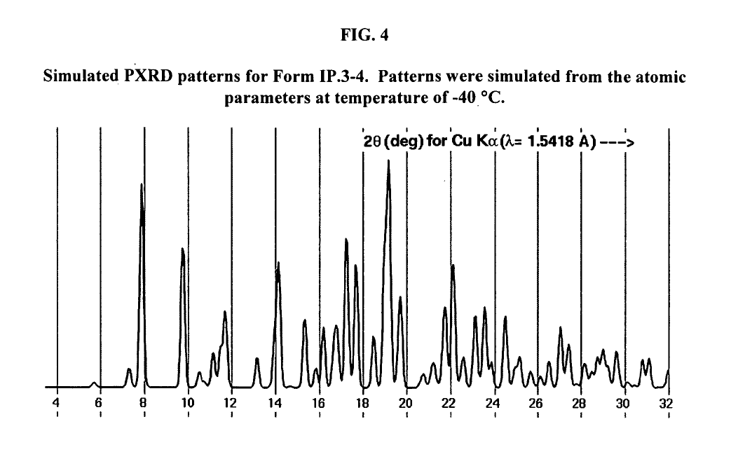

In yet another aspect, there is provided a crystalline form of [1S-(1α,3α,4β)]-2-amino-1,9-dihydro-9-[4-hydroxy-3-(hydroxymethyl)-2-methylenecyclopentyl]-6H-purin-6-one, designated as Form IP.3-4, which exhibits an PXRD pattern substantially the same as that depicted in FIG. 4.

In a further aspect, the invention provides a process for preparing the aforementioned Form N-2 crystals, which process comprises crystallizing [1S-(1α,3α,4β)]-2-amino-1,9-dihydro-9-[4-hydroxy-3-(hydroxymethyl)-2-methylenecyclopentyl]-6H-purin-6-one from an anhydrous organic solvent, such as methanol.

In yet another aspect, the invention provides a process for preparing the aforementioned Form IP.3-4 crystals, which process comprises crystallizing [1S-(1α,3α,4β)]-2-amino-1,9-dihydro-9-[4-hydroxy-3-(hydroxymethyl)-2-methylenecyclopentyl]-6H-purin-6-one from isopropanol.

Table 5 further exemplifies processes for the preparation of Form N-2 and Form IP.3-4 crystals of [1S-(1α,3α,4β)]-2-amino-1,9-dihydro-9-[4-hydroxy-3-(hydroxymethyl)-2-methylenecyclopentyl]-6H-purin-6-one.

TABLE 5
Crystallization Solvents and Morphologies

| Form | Recrystallization solvents | Habit |
|---|---|---|
| N-2 | MeOH @ ˜55° C. | large tetragonal prisms |
| IP.3-4 | Isopropanol @ ˜70° C. | rectangular plates |

In a further aspect, the invention provides a process for preparing a pharmaceutical composition comprising [1S-(1α,3α,4β)]-2-amino-1,9-dihydro-9-[4-hydroxy-3-(hydroxymethyl)-2-methylenecyclopentyl]-6H-purin-6-one and at least one pharmaceutically acceptable carrier or excipient, which process comprises mixing Form N-2 and/or IP.3-4 crystals of [1S-(1α,3α,4β)]-2-amino-1,9-dihydro-9-[4-hydroxy-3-(hydroxymethyl)-2-methylenecyclopentyl]-6H-purin-6-one with at least one said pharmaceutically acceptable carrier or excipient. Preferred processes comprise mixing the aforementioned Form N-2 crystals with at least one pharmaceutically acceptable carrier or excipient. Pharmaceutically acceptable carriers or excipients include, without limitation, polyether glycols, saturated or unsaturated polyglycolized glyceridea, solid amphiphilic surfactants, surfactants other than said solid amphiphilic surfactants, alcohols other than a polyether glycols, fatty acid ester derivatives of polyhydric alcohols, vegetable oils, mineral oils, and optionally, an effective amount of a pharmaceutically acceptable acid for enhancing the stability of the drug. It should be understood that the crystalline forms of Form N-2 and Form IP.3-4 may, in some cases, change to other form or forms (e.g., amorphous), or solubilize, upon mixing with at least one pharmaceutically acceptable carrier or excipient.

In a yet another aspect, the invention provides methods for treating a patient infected with hepatitis B virus infection or co-infected with hepatitis B and another viral or non-viral disease, which method comprises administering to the patient crystals of Form N-2, Form IP.3-4, or mixtures thereof, or a pharmaceutical composition comprising crystals of Form N-2 crystals, Form IP.3-4 crystals, or a mixture thereof. A preferred crystal form useful in the practice of the instant methods of treating a patient infected with hepatitis B virus infection or co-infected with hepatitis B and another viral or non-viral disease comprises [1S-(1α,3α,4β)]-2-amino-1,9-dihydro-9-[4-hydroxy-3-(hydroxymethyl)-2-methylenecyclopentyl]-6H-purin-6-one Form N-2 crystals and the preferred method of administering to a patient using such Form N-2 crystals is oral on a daily basis.

In a yet another aspect, the invention provides pharmaceutical compositions comprising Form N-2 and/or Form IP.3-4 crystals of [1S-(1α,3α,4β)]-2-amino-1,9-dihydro-9-[4-hydroxy-3-(hydroxymethyl)-2-methylenecyclopentyl]-6H-purin-6-one and at least one pharmaceutically acceptable carrier or excipient. The pharmaceutical compositions may contain a low dose of from about 0.001 mg to about 25 mg of the active drug ingredient for once daily administration to treat hepatitis B virus infection in an adult human patient or a pediatric patient. Preferred pharmaceutical compositions contain from about 0.01 mg to about 10 mg of the active drug ingredient and most preferred pharmaceutical compositions contain from about 0.01 to about 5 mg of the active drug ingredient. Such preferred and most preferred pharmaceutical compositions are also administered once daily to treat hepatitis B virus infection in an adult or a pediatric patient.

The term adult human patient is defined as a patient of about 16 years or more of age and a weight equal to or greater than about 50 kilograms. Pharmaceutical compositions containing the active drug ingredient at the lower end of the above ranges are suitable for administration to pediatric patients or adult patients weighing less than about 50 kilograms.

The low dose pharmaceutical compositions described above for daily administration may also be administered to certain patients less often. For example, patients who have been treated by daily administration of the low dose pharmaceutical compositions so that their hepatitis B virus infection is now under control may be placed on a maintenance regimen to protect against further infection. Such maintenance therapy may involve the administration of the low dose composition on a less than daily basis. For example, a single dose administered every three or four days or administered on a weekly basis may be sufficient.

The low dose pharmaceutical compositions as mentioned above can be formulated for administration by any suitable means. For example, compositions for oral administration, which are preferred, can be in the form of tablets, capsules, granules or powders or in the form of elixirs, solutions or suspensions. The low dose pharmaceutical compositions may also be formulated for parenteral, rectal, transdermal or nasal administration according to methods well known in the art. Such formulations can include pharmaceutically acceptable excipients including bulking agents, lubricants, disintegrants, binding agents, etc. as commonly employed in such compositions. Sustained release formulations are also within the scope of this invention.

Solid dosage forms for oral administration include capsules, tablets, powders, and granules. In such dosage forms, the crystal forms of the instant invention are preferably admixed with at least one inert customary pharmaceutical excipient (or carrier) such as sodium citrate, or dicalcium phosphate, or (a) fillers or extenders; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, cetyl alcohol and glycerol monostearate; (g) adsorbents, as for example, kaolin and bentonite; and/or (h) lubricants, as for example, talc, calcium stearate, magnesium stearate, solid polyethylene glycols, sodium lauryl sulfate, or mixtures thereof. In the case of capsules and tablets, the dosages forms may also comprise buffering agents.

Solid compositions of a similar type may also be employed as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coating and shells such as enteric coatings and others well known in the art. They may also contain certain opacifying agents, and can be of such composition that they release the active compound or compounds in a delayed manner. Examples of embedding compositions that can also be employed are polymeric substances and waxes. The crystal forms of the instant invention can also be incorporated in micro-encapsulated form, if appropriate, with one or more of the above-mentioned excipients.

Liquid dosage forms for oral administration include, for example, pharmaceutically acceptable emulsions, solutions, suspensions, syrups, and elixirs. In addition to the crystal forms of the instant invention, the liquid dosage form may contain inert diluents such as those commonly used in the art, e.g., water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oil, in particular, cottonseed oil, groundnut oil, corn germ oil, castor oil, and sesame seed oil, glycerol, tetrahydrofurfuryl alcohol, polyethylene glycols, and fatty acid esters of sorbitan, or mixtures of these substances, and the like.

Besides such inert diluents, the compositions may also comprise adjuvants, such as wetting agents, emulsifying and suspending agents, sweetening, flavoring, and perfuming agents.

Suspensions of the crystal forms of the instant invention may further comprise suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by admixing the crystal forms of the instant invention with suitable non-irritating excipients or carriers such as cocoa butter, polyethylene glycol, or a suppository wax, which are solid at room temperature, but liquid at body temperature and, therefore, melt in the rectum or vaginal cavity thereby releasing such crystal forms.

Dosage forms for topical administration may comprise ointments, powders, sprays, and inhalants. The crystal forms of the instant invention are admixed under sterile conditions with a pharmaceutically acceptable carrier, and any preservatives, buffers, or propellants that may also be required. Opthalmic formulations, eye ointments, powders, and solutions are also intended to be included within the scope of the present invention.

It has been found that once daily administration of the low dose entecavir pharmaceutical compositions are effective in treating hepatitis B virus infection without undesirable side effects that can result from administration of the high dose regimen described in U.S. Pat. No. 5,206,244.

The low dose compositions as described above can be used to treat hepatitis B virus infection in combination with one or more other pharmaceutically active agents. Suitable pharmaceutically active agents for this purpose include one or more antiviral agents, for example, didanosine, lamivudine, abacavir, adefovir, adefovir dipivoxil, famciclovir, (2R,4R)-4-(2,6-diamino-9H-purin-9-yl)-2-hydroxymethyl-1,3-dioxolane (DAPD), hepatitis B immunomodulating proteins (EHT 899 from Enzo Biochem), emtricitabine, 1-(2-deoxy-2-fluoro-β-D-arabinofuranosyl)thymine(FMAU), GLQ-223 (Compound A, alpha-trichosanthin), epavudine (L-dT), epcitabine (L-dC), ribavirin, tenofovir (PMPA), 2′,3′-dideoxy-2′,3′-didehydro-beta-L(−)-5-fluorocytidine[L(−)Fd4C], as well as other fluoro L- and D-nucleosides. Suitable pharmaceutically active agents for this purpose also include one or more immunomodulators, for example, alpha interferon, beta interferon, pegylated interferon, thymosin alpha, and hepatitis B vaccines such as HBV/MF59, Hepagene and Theradigm-HBV.

When the other pharmaceutically active agent or agents are suitable for oral administration, they can be combined with the low dose of Form N-2 and/or IP.3-4 into a single tablet or capsule. If the other pharmaceutically active agent or agents are not compatable with the Form N-2 and/or IP.3-4 for co-administration from a single dosage form, for example, if the mode of administration is different or if the frequency of administration is different, then the other pharmaceutically active agent or agents will be administered separately. The amount of the other agent or agents administered is that conventionally employed in mono therapy or a reduced amount as determined by the treating physician. The separate dose forms can be administered at the same time or sequentially according to a prescribed schedule.

The low dose compositions as described above can also be used to treat co-infected patients. A co-infected patient is one infected with other viral or non-viral diseases in addition to hepatitis B. In particular, such treatment is possible for hepatitis B patients co-infected with hepatitis C or HIV. Such co-infected patients are preferably treated with the low dose [1S-(1α,3α,4β)]-2-amino-1,9-dihydro-9-[4-hydroxy-3-(hydroxymethyl)-2-methylenecyclopentyl]-6H-purin-6-one compositions as described above in combination with one or more other pharmaceutically active agents as described above. For example, a patient co-infected with hepatitis B and hepatitis C can be treated with the low dose composition in addition to being treated with a regimen of ribavirin and an interferon.

Tablet and capsule formulations containing from about 0.001 mg to about 10 mg of crystals of Form N-2 and/or IP.3-4 can be prepared according to the following procedures that ensure high potency and good uniformity of the product. The compositions can be prepared by first carefully depositing the active drug ingredient on the surface of carrier substrate particles. This step is accomplished by forming a solution of the [1S-(1α,3α,4β)]-2-amino-1,9-dihydro-9-[4-hydroxy-3-(hydroxymethyl)-2-methylenecyclopentyl]-6H-purin-6-one in an appropriate solvent along with an adhesive substance at temperatures ranging from about 25° C. to about 80° C. and applying the solution as a spray or a stream while the carrier substrate particles are in motion. The conditions are controlled to minimize particle agglomeration. Subsequently, the solvent is removed from the carrier surface leaving the drug particles adhered to the surface of the carrier substrate. This prevents the separation of the active drug ingredient from the substrate and minimizes the loss of the drug during subsequent processing.

Following drying, the drug coated carrier substrate particles are mixed with any other ingredients to be included in the composition such as a disintegrant and/or lubricant. The resulting powder is then compressed into tablets or filled into capsules.

The carrier substrate particles are kept in motion during the spraying step by means of mechanical or air stream agitation. In the mechanical agitation procedure, the carrier substrate is placed in a mechanical (high shear) mixer and agitated. A solution containing the active drug ingredient and adhesive substance maintained at a temperature of from about 25° C. to about 80° C. is sprayed onto the carrier substrate particles at a controlled rate and atomizing pressure (0 to 2 bar). To maximize the amount of drug deposited on the carrier, the position of the spray assembly is adjusted to make certain that the spray pattern only encompasses the carrier. The rate of deposition and the spray pattern are controlled to minimize particle agglomeration. Once the drug containing solution is deposited, the wet drug/carrier substrate particles are transferred to a drier, either a tray drier or fluidbed drier is suitable. The solvent is removed at an elevated temperature. When the solvent is water or pH adjusted water, a temperature of from about 50° to about 80° C. is suitable.

In the air stream agitation procedure, the carrier substrate is placed in a bowl with a fine mesh screen at the bottom. The incoming air stream is adjusted so that the substrate particle motion is constant and fluid. The carrier material is equilibrated to a temperature of from about 25° C. to about 80° C. A solution containing the active drug ingredient and adhesive substance maintained at a temperature of from about 25° C. to about 80° C. is sprayed onto the carrier substrate particles at a controlled rate and atomizing pressure as described above. Again, the position of the spray assembly is adjusted to make certain that the spray pattern only encompasses the carrier and the rate of deposition is controlled to minimize particle agglomeration. Once the drug solution is deposited, the temperature is elevated to remove the solvent. When the solvent is water or pH adjusted water, a temperature of from about 50° C. to about 80° C. is suitable. In the air stream agitation procedure, both the deposition of the drug onto the carrier substrate and the removal of the solvent are carried out in a single unit whereas the mechanical agitation procedure requires a two-unit operation.

The adhesive substance is preferably a polymeric material possessing a high degree of tackiness. Suitable materials include povidone, methylcellulose, hydroxymethylcellulose, hydroxypropylmethylcellulose, hydroxypropylcellulose, hydroxyethylcellulose, gelatin, guar gum, and xanthan gum and mixtures thereof with povidone being preferred. The adhesive substance is preferably present in the final composition at from about 0.01% to about 10% by weight of the total composition.

The carrier substrate is a pharmaceutically acceptable substance that can be readily spray coated and yet will not easily agglomerate. Suitable materials include lactose, microcrystalline cellulose, calcium phosphate, dextrin, dextrose, dextrates, mannitol, sorbitol, and sucrose and mixtures thereof with lactose and microcrystalline cellulose and mixtures thereof being preferred. The carrier substrate is preferably present in the final composition at from about 80% to about 95% by weight of the total composition.

A disintegrant is preferably included in the final composition at from about 1% to about 7% by weight of the total composition. Suitable disintegrants include crospovidone, croscarmellose, sodium starch glycolate, pregelatinized starch, and corn starch and mixtures thereof with crospovidone being preferred.

A lubricant is preferably included in the final composition at from about 0.1% to about 5% by weight of the total composition. Suitable lubricants include magnesium stearate, stearic acid, sodium stearyl fumarate, and sodium lauryl sulfate with magnesium stearate being preferred.

The resulting tablet or capsule can be film coated for ease of administration. Suitable materials for use in the film coating are polymeric coating agents, pigments, plasticizers, solubilizing agents, etc. Suitable coating agents include hydroxypropyl methylcellulose, hydroxyethyl cellulose, hydroxypropyl cellulose, hydroxypropyl methylcellulose phthalate, etc. Polyethylene glycol can be included in the film coating composition as a plasticizer. Additional plasticizers such as diethyl citrate and trietyl citrate may also be included in the film coating composition. Suitable solubilizing agents include polyoxyethylene sorbitan fatty acid esters particularly polysorbate 80. Suitable pigments include titanium dioxide and various iron oxides.

The ingredients of the coating compositions are dispersed in a suitable solvent, preferably water. The coating composition can be applied to the tablets or capsules using conventional pan coating or spray coating techniques.

Employing the above procedures, a tablet of 0.1 milligram strength of [1S-(1α,3α,4β)]-2-amino-1,9-dihydro-9-[4-hydroxy-3-(hydroxymethyl)-2-methylenecyclopentyl]-6H-purin-6-one can be prepared using crystals of Form N-2 and/or Form IP.3-4 as in Table 6:

TABLE 6

| | Amount | |
|---|---|---|
| | % weight/ | Amount |
| Ingredient | weight | per capsule |
| 1S-(1α, 3α, 4β)]-2-amino-1,9-dihydro-9-[4- | 0.1 | 0.1 mg |
| hydroxy-3-(hydroxymethyl)-2- | | |
| methylenecyclopentyl]-6H-purin-6-one | | |
| Lactose monohydrate, NF | 60.00 | 60.00 |
| Microcrystalline | 35.39 | 35.39 mg |
| cellulose, NF | | |
| Crospovidone, NF | 4.0 | 4.00 mg |
| Povidone, USP | 0.01 | 0.01 mg |
| Magnesium Stearate, NF | 0.5 | 0.5 mg |
| Purified Water, USP* | q.s. | — |
| Total | 100.00 | 100.00 mg |

### Experimental

Single Crystal Data

Data were collected on a Bruker-Nonius¹ CAD4 serial diffractometer. Unit cell parameters were obtained through least-squares analysis of the experimental diffractometer settings of 25 high-angle reflections. Intensities were measured using Cu Kα radiation (k=1.5418 Å) at a constant temperature with the 0-20 variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (k=1.5418 Å). Indexing and processing of the measured intensity data were carried out with the HKL2000-software package² in the Collect program suite.³
¹ BRUKER AXS, Inc., 5465 East Cheryl Parkway Madison, Wis. 53711 USA

² Otwinowski, Z. & Minor, W. (1997) in Macromolecular Crystallography, eds. Carter, W. C. Jr & Sweet, R. M. (Academic, NY), Vol. 276, pp. 307-326

³ Collect Data collection and processing user interface: Collect: Data collection software, R. Hooft, Nonius B. V., 1998

When indicated, crystals were cooled in the cold stream of an Oxford cryo system⁴ during data collection.
⁴ Oxford Cryosystems Cryostream cooler: J. Cosier and A. M. Glazer, J. Appl. Cryst., 1986, 19, 105.

The structures were solved by direct methods and refined on the basis of observed reflections using either the SDP⁵ software package with minor local modifications or the crystallographic package, MAXUS.⁶
⁵SDP, Structure Determination Package, Enraf-Nonius, Bohemia N.Y. 11716 Scattering factors, including f′ and f″, in the SDP software were taken from the “International Tables for Crystallography”, Kynoch Press, Birmingham, England, 1974; Vol IV, Tables 2.2A and 2.3.1

⁶ maXus solution and refinement software suite: S. Mackay, C. J. Gilmore, C. Edwards, M. Tremayne, N. Stewart, K. Shankland. maXus: a computer program for the solution and refinement of crystal structures from diffraction data

The derived atomic parameters (coordinates and temperature factors) were refined through full matrix least-squares. The function minimized in the refinements was Σ_{W}(|F_{O}|−|_{C}|)²·R is defined as Σ||F_{O}|−|F_{C}||/Σ|F_{O}| while R_{W}=[Σ_{W}(|F_{O}|−|F_{C}|)²/Σ_{W}|F_{O}|²]^{1/2} where w is an appropriate weighting function based on errors in the observed intensities. Difference maps were examined at all stages of refinement. Hydrogens were introduced in idealized positions with isotropic temperature factors, but no hydrogen parameters were varied.

Powder X-Ray Diffraction (PXRD)

About 200 mg were pack by the backloading method into a Philips powder X-ray diffraction (PXRD) sample holder. The sample was transferred to a Philips MPD unit (45 KV, 40 mA, Cu Kα). Data were collected at room temperature in the 2 to 32 2-theta range (continuous scanning mode, scanning rate 0.03 degrees/sec., auto divergence and anti scatter slits, receiving slit: 0.2 mm, sample spinner: ON)

The simulated PXRD may be calculated from single crystal x-ray data. See Smith, D. K., “A FORTRAN Program for Calculating X-Ray Powder Diffraction Patterns,” Lawrence Radiation Laboratory, Livermore, Calif., UCRL-7196 (April 1963).

Differential Scanning Calorimetry (DSC)

Differential scanning calorimetry (DSC) experiments were performed in a TA Instruments™ model Q1000 or 2920. The sample (about 2-6 mg) was weighed in an aluminum pan and recorded accurately recorded to a hundredth of a milligram, and transferred to the DSC. The instrument was purged with nitrogen gas at 50 mL/min. Data were collected between room temperature and 350° C. at 110° C./min heating rate. The plot was made with the endothermic peaks pointing down.

Thermogravimetric Analysis (TGA)

Thermal gravimetric analysis (TGA) experiments were performed in a TA Instruments™ model Q500 or 2950. The sample (about 10-30 mg) was placed in a platinum pan previously tared. The weight of the sample was measured accurately and recorded to a thousand of a milligram by the instrument The furnace was purged with nitrogen gas at 100 mL/min. Data were collected between room temperature and 350° C. at 10° C./min heating rate.

We claim:

1. A crystalline form of [1S-(1α,3α,4β)]-2-amino-1,9-dihydro-9-[4-hydroxy-3-(hydroxymethyl)-2-methylenecyclopentyl]-6H-purin-6-one represented by formula I: 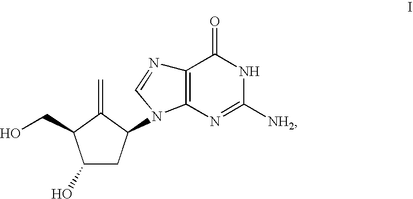
comprising Form N-2 crystals characterized by unit cell parameters approximately equal to the following:

| Cell dimensions | a = 7.524(1) Å, |
|---|---|
| | b = 7.524(1) Å, |
| | c = 43.970(5) Å, |
| | Volume = 2489(1) A³, |
| Space group | P43212, |
| Molecules/unit cell | 8, |
| Density (calculated) (g/cm³) | 1.480; |

and said Form N-2 crystals consisting of one molecule of formula I per asymmetric unit in an arrangement and conformation according to the fractional atomic coordinates expressed in Table 2.
TABLE 2

| Atom | X | Y | Z |
|---|---|---|---|
| O1 | 0.667054 | 0.068305 | −0.106759 |
| O2 | 0.571618 | 0.116352 | 0.099202 |
| N3 | 0.662231 | 0.651071 | −0.077612 |
| N4 | 0.700851 | 0.193505 | −0.004899 |
| N5 | 0.671599 | 0.354600 | −0.090095 |
| N6 | 0.689785 | 0.448266 | −0.038502 |
| N7 | 0.703595 | −0.030067 | −0.038659 |
| O8 | 0.966911 | 0.087121 | 0.119548 |
| C9 | 0.696989 | 0.132596 | −0.053543 |
| C10 | 0.676487 | 0.483243 | −0.067785 |
| C11 | 0.679224 | 0.171396 | −0.084865 |
| C12 | 0.703921 | 0.285220 | 0.024643 |
| C13 | 0.696218 | 0.271804 | −0.033061 |
| C14 | 0.706100 | 0.014087 | −0.009683 |
| C15 | 0.567168 | 0.21124 | 0.047311 |
| C16 | 0.882274 | 0.264152 | 0.040554 |
| C17 | 0.675486 | 0.119954 | 0.072150 |
| C18 | 0.848985 | 0.225864 | 0.073877 |
| C19 | 1.004899 | 0.131649 | 0.088687 |
| C20 | 1.035968 | 0.268005 | 0.026482 |
| H21 | 0.7132 | −0.0821 | 0.0086 |
| H22 | 0.6630 | 0.3981 | −0.1134 |
| H23 | 0.6772 | 0.4237 | 0.0210 |
| H24 | 0.4897 | 0.3183 | 0.0568 |
| H25 | 0.4805 | 0.1175 | 0.0362 |
| H26 | 0.7059 | −0.0144 | 0.0651 |
| H27 | 0.8313 | 0.3467 | 0.0868 |
| H28 | 1.1205 | 0.2170 | 0.0879 |
| H29 | 1.0334 | 0.0102 | 0.0763 |
| H30 | 1.1587 | 0.2518 | 0.0396 |
| H31 | 1.0436 | 0.2844 | 0.0023 |
| H32 | 0.9959 | 0.1920 | 0.1330 |
| H33 | 0.6151 | 0.0163 | 0.1127 |
| H34 | 0.6810 | 0.7522 | −0.0637 |
| H35 | 0.6712 | 0.6813 | −0.0991 |

2. The crystalline form of claim 1 which is substantially pure.

3. A crystalline form of [1S-(1α,3α,4β)]-2-amino-1,9-dihydro-9-[4-hydroxy-3-(hydroxymethyl)-2-methylenecyclopentyl]-6H-purin-6-one (Form N-2) which exhibits a powder X-ray diffraction pattern comprising 2θ values selected from: 11.9±0.2, 16.1±0.2, 16.8±0.2, 17.2±0.2, 20.0±0.2, 23.7±0.2, 24.0±0.2, 24.4±0.2, and 25.0±0.2.

4. A crystalline form of [1S-(1α,3α,4β)]-2-amino-1,9-dihydro-9-[4-hydroxy-3-(hydroxymethyl)-2-methylenecyclopentyl]-6H-purin-6-one (Form N-2) which exhibits a powder X-ray diffraction pattern substantially the same as shown in FIG. 1.

5. A crystalline form of [1S-(1α,3α,4β)]-2-amino-1,9-dihydro-9-[4-hydroxy-3-(hydroxymethyl)-2-methylenecyclopentyl]-6H-purin-6-one (Form N-2) which exhibits a DSC thermogram substantially the same as shown in FIG. 2.

6. A crystalline form of [1S-(1α,3α,4β)]-2-amino-1,9-dihydro-9-[4-hydroxy-3-(hydroxymethyl)-2-methylenecyclopentyl]-6H-purin-6-one (Form N-2) which exhibits a TGA thermogram substantially the same as shown in FIG. 3.

7. A crystalline form of [1S-(1α,3α,4β)]-2-amino-1,9-dihydro-9-[4-hydroxy-3-(hydroxymethyl)-2-methylenecyclopentyl]-6H-purin-6-one represented by formula I: 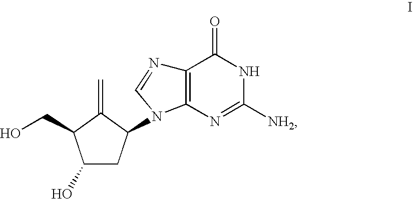
comprising Form IP.3-4 crystals characterized by unit cell parameters approximately equal to the following:

| Cell dimensions | a = 12.612(2) Å, |
|---|---|
| | b = 11.199(2) Å, |
| | c = 16.070(2) Å, |
| | β = 106.72(1) °, |
| | Volume = 2173.8(1) A³, |
| Space group | P21, |
| Molecules of Formula I/unit cell | 6, |
| Molecules of Isopropanol/unit cell | 2, |
| Density (calculated) (g/cm³) | 1.363; |

and said Form IP.3-4 crystals consisting of three molecules of formula I and one molecules of isopropanol per asymmetric unit in an arrangement and conformation according to the fractional atomic coordinates expressed in Table 4.
TABLE 4

| Atom | X | Y | Z |
|---|---|---|---|
| O1 | 0.3455 | 0.6778 | 0.3033 |
| C2 | 0.0971 | 0.8297 | 0.2553 |
| C3 | 0.0945 | 0.4982 | 0.2657 |
| O4 | 0.4077 | −0.0863 | 0.2953 |
| C5 | 0.0098 | 0.3749 | 0.4934 |
| C6 | 0.4434 | −0.0558 | 0.3774 |
| C7 | 0.1602 | 0.1368 | 0.4350 |
| C8 | 0.6360 | 0.4386 | 0.0954 |
| C9 | −0.0250 | 0.6269 | 0.3292 |
| O10 | 0.1749 | 0.9083 | 0.2603 |
| N11 | 0.0629 | 0.4835 | 0.5175 |
| C12 | 0.1652 | 0.5098 | 0.5155 |
| O13 | 0.5976 | 0.2041 | 0.5038 |
| C14 | 0.5219 | 0.1093 | 0.4895 |
| C15 | 0.3650 | 0.1595 | 0.3674 |
| C16 | 0.2919 | 0.1657 | 0.2889 |
| C17 | 0.3520 | 0.2177 | 0.4488 |
| C18 | 0.4231 | 0.1415 | 0.5217 |
| C19 | 0.4744 | 0.0886 | 0.3920 |
| O20 | 0.7306 | 0.4833 | 0.3779 |
| C21 | 0.6760 | 0.3810 | 0.3297 |
| N22 | 0.2369 | 0.2279 | 0.4498 |
| N23 | 0.0662 | 0.1708 | 0.4479 |
| C24 | 0.5539 | 0.4096 | 0.2945 |
| N25 | 0.2026 | 0.6241 | 0.5374 |
| O26 | −0.0880 | 0.3590 | 0.4930 |
| C27 | 0.0828 | 0.2888 | 0.4728 |
| C28 | 0.1882 | 0.3246 | 0.4742 |
| N29 | 0.2360 | 0.4344 | 0.4939 |
| N30 | 0.0234 | 0.4844 | 0.1758 |
| C31 | −0.0158 | 0.3780 | 0.1328 |
| N32 | −0.0874 | 0.5337 | 0.0450 |
| N33 | 0.0105 | 0.2672 | 0.1660 |
| N34 | −0.0242 | 0.0669 | 0.1321 |
| C35 | −0.0412 | 0.1866 | 0.1100 |
| C36 | −0.0828 | 0.4106 | 0.0543 |
| N37 | −0.1145 | 0.2098 | 0.0303 |
| C38 | −0.0237 | 0.5739 | 0.1182 |
| C39 | −0.1404 | 0.3214 | −0.0044 |
| O40 | −0.2074 | 0.3355 | −0.0767 |
| C41 | 0.2157 | 0.5132 | 0.2724 |
| C42 | 0.2302 | 0.6484 | 0.2627 |
| C43 | 0.0625 | 0.6128 | 0.3034 |
| C44 | 0.1535 | 0.7064 | 0.3102 |
| C45 | 0.3628 | 0.5669 | 0.1166 |
| N46 | 0.2676 | 0.5336 | 0.0629 |
| C47 | 0.3741 | 0.3720 | 0.1133 |
| C48 | 0.2733 | 0.4099 | 0.0630 |
| N49 | 0.4105 | 0.2550 | 0.1268 |
| N50 | 0.4314 | 0.4720 | 0.1485 |
| N51 | 0.2292 | 0.2100 | 0.0390 |
| C52 | 0.3323 | 0.1771 | 0.0889 |
| N53 | 0.3537 | 0.0602 | 0.0972 |
| C54 | 0.1929 | 0.3280 | 0.0189 |
| O55 | 0.1007 | 0.3445 | −0.0324 |
| C56 | 0.5430 | 0.4787 | 0.2122 |
| C57 | 0.6312 | 0.4272 | 0.1758 |
| C58 | 0.7152 | 0.3599 | 0.2498 |
| O59 | 0.6204 | 0.1671 | 0.2213 |
| C60 | 0.7228 | 0.2268 | 0.2279 |
| O61 | 0.5656 | −0.0619 | 0.2058 |
| C62 | 0.6336 | −0.1557 | 0.1981 |
| C63 | 0.6242 | −0.1943 | 0.1186 |
| C64 | 0.7391 | −0.1449 | 0.2523 |
| H65 | −0.0680 | 0.0006 | 0.0856 |
| H66 | 0.0305 | 0.0403 | 0.1933 |
| H67 | −0.0090 | 0.6699 | 0.1329 |
| H68 | −0.1535 | 0.1344 | −0.0095 |
| H69 | 0.0835 | 0.4191 | 0.3018 |
| H70 | 0.2681 | 0.4818 | 0.3337 |
| H71 | 0.2368 | 0.4656 | 0.2200 |
| H72 | 0.2058 | 0.6741 | 0.1948 |
| H73 | 0.0355 | 0.8648 | 0.2858 |
| H74 | 0.0578 | 0.8083 | 0.1898 |
| H75 | 0.5620 | 0.0314 | 0.5230 |
| H76 | 0.3073 | 0.1211 | 0.2340 |
| H77 | 0.2168 | 0.2190 | 0.2789 |
| H78 | 0.5367 | 0.1128 | 0.3586 |
| H79 | 0.3819 | −0.0763 | 0.4091 |
| H80 | 0.5185 | −0.1070 | 0.4075 |
| H81 | 0.3835 | 0.3090 | 0.4537 |
| H82 | 0.6864 | 0.3045 | 0.3718 |
| H83 | 0.7458 | 0.2209 | 0.1672 |
| H84 | 0.7906 | 0.1862 | 0.2789 |
| H85 | 0.7984 | 0.3976 | 0.2606 |
| H86 | 0.5056 | 0.3281 | 0.2798 |
| H87 | 0.5262 | 0.4609 | 0.3414 |
| H88 | 0.5601 | 0.5739 | 0.2305 |
| H89 | 0.3786 | 0.0616 | 0.5307 |
| H90 | 0.4484 | 0.1900 | 0.5823 |
| H91 | 0.1763 | 0.0473 | 0.4139 |
| H92 | −0.0431 | 0.7111 | 0.3567 |
| H93 | −0.0827 | 0.5525 | 0.3269 |
| H94 | 0.2024 | 0.7306 | 0.3758 |
| H95 | 0.3856 | 0.6614 | 0.1340 |
| H96 | 0.5525 | 0.2193 | 0.1812 |
| H97 | 0.8047 | 0.4538 | 0.4273 |
| H98 | 0.1574 | 0.6889 | 0.5258 |
| H99 | 0.2646 | 0.6471 | 0.5196 |
| H100 | 0.7024 | 0.3988 | 0.0744 |
| H101 | 0.5719 | 0.4887 | 0.0472 |
| H102 | 0.6094 | 0.0209 | 0.2037 |
| H103 | 0.4743 | −0.0771 | 0.2648 |
| H104 | 0.6329 | 0.2162 | 0.5740 |
| H105 | 0.2017 | 0.9020 | 0.2027 |
| H106 | 0.3576 | 0.7725 | 0.2962 |
| H107 | 0.0173 | 0.5526 | 0.5406 |
| H108 | 0.2893 | −0.0043 | 0.0665 |
| H109 | 0.4339 | 0.0279 | 0.1343 |
| H110 | 0.1709 | 0.1388 | 0.0114 |
| H113 | 0.6813 | −0.2671 | 0.1215 |
| H112 | 0.5406 | −0.2250 | 0.0891 |
| H114 | 0.6430 | −0.1225 | 0.0803 |
| H115 | 0.7880 | −0.2203 | 0.2434 |
| H117 | 0.7758 | −0.0634 | 0.2375 |
| H116 | 0.7365 | −0.1423 | 0.3189 |
| H111 | 0.6158 | −0.2298 | 0.2349 |

8. The crystalline form of claim 7 which is substantially pure.

9. A crystalline form of [1S-(1α,3α,4β)]-2-amino-1,9-dihydro-9-[4-hydroxy-3-(hydroxymethyl)-2-methylenecyclopentyl]-6H-purin-6-one (Form IPA.3-4) which exhibits a powder X-ray diffraction pattern substantially the same as shown in FIG. 4.

10. A process for preparing Form N-2 crystals of [1S-(1α,3α,4β)]-2-amino-1,9-dihydro-9-[4-hydroxy-3-(hydroxymethyl)-2-methylenecyclopentyl]-6H-purin-6-one, which process comprises crystallizing [1S-(1α,3α,4β)]-2-amino-1,9-dihydro-9-[4-hydroxy-3-(hydroxymethyl)-2-methylenecyclopentyl]-6H-purin-6-one from an anhydrous organic solvent.

11. The process of claim 10, wherein said organic solvent is methanol.

12. A process for preparing Form IP.3-4 crystals of [1S-(1α,3α,4β)]-2-amino-1,9-dihydro-9-[4-hydroxy-3-(hydroxymethyl)-2-methylenecyclopentyl]-6H-purin-6-one, which process comprises crystallizing [1S-(1α,3α,4β)]-2-amino-1,9-dihydro-9-[4-hydroxy-3-(hydroxymethyl)-2-methylenecyclopentyl]-6H-purin-6-one from isopropanol.

13. A process for preparing a pharmaceutical formulation containing [1S-(1α,3α,4β)]-2-amino-1,9-dihydro-9-[4-hydroxy-3-(hydroxymethyl)-2-methylenecyclopentyl]-6H-purin-6-one and at least one pharmaceutically acceptable carrier or excipient, which process comprises mixing Form N-2 crystals of [1S-(1α,3α,4β)]-2-amino-1,9-dihydro-9-[4-hydroxy-3-(hydroxymethyl)-2-methylenecyclopentyl]-6H-purin-6-one with at least one said pharmaceutically acceptable carrier or excipient.

14. A pharmaceutical composition comprising Form N-2 and/or Form IP.3-4 crystals of [1S-(1α,3β,4β)]-2-amino-1,9-dihydro-9-[4-hydroxy-3-(hydroxymethyl)-2-methylenecyclopentyl]-6H-purin-6-one and at least one pharmaceutically acceptable carrier or excipient.

15. A method for treating a patient infected with hepatitis B virus infection or co-infected with hepatitis B and another viral or non-viral disease, which method comprises administering to the patient Form N-2 and/or a Form IP.3-4 crystals of [1S-(1α,3α,4β)]-2-amino-1,9-dihydro-9-[4-hydroxy-3-(hydroxymethyl)-2-methylenecyclopentyl]-6H-purin-6-one, or a pharmaceutical composition containing such Form N-2 crystals, Form IP.3-4 crystals, or a mixture thereof.

16. The method of claim 15, which method comprises administering a pharmaceutical composition containing from about 0.001 mg to about 25 mg of Form N-2 crystals, Form IP.3-4 crystals, or a mixture thereof on daily basis.

17. The method of claim 16, wherein said pharmaceutical composition contains from about 0.01 mg to about 10 mg of Form N-2 crystals of [1S-(1α,3α,4β)]-2-amino-1,9-dihydro-9-[4-hydroxy-3-(hydroxymethyl)-2-methylenecyclopentyl]-6H-purin-6-one.
